(19) United States
(12) Patent Application Publication
(10) Pub. No.: US 2025/0231180 A1
(43) Pub. Date: Jul. 17, 2025

(54) FLUORESCENT TURN-ON CHEMOSENSORS FOR DETECTION OF ALUMINUM ION AND AZIDE

(71) Applicant: MORGAN STATE UNIVERSITY, Baltimore, MD (US)

(72) Inventor: Fasil Abebe, Elkridge, MD (US)

(21) Appl. No.: 19/091,216

(22) Filed: Mar. 26, 2025

Related U.S. Application Data
(62) Division of application No. 18/299,710, filed on Apr. 12, 2023, now Pat. No. 12,287,326.
Application No. 18/299,710 is a continuation of application No. 16/987,688, filed on Aug. 7, 2020, now abandoned.
(60) Provisional application No. 62/883,785, filed on Aug. 7, 2019.

(51) Int. Cl. G01N 33/52; C07D 311/88; C07D 491/107; C07F 5/06; G01N 21/78

(52) U.S. Cl. CPC G01N 33/52; C07D 311/88; C07D 491/107; C07F 5/069; G01N 21/78

(57) ABSTRACT

Two rhodamine reversible fluorescent sensor derivatives, L₁ and L₂, bearing 2-methoxy-1-naphthaldehyde and 5-bromo-3-methoxy salicylaldehyde units were synthesized using microwave-assisted organic synthesis and used for selective and sensitive reversible sequential fluorescence detection of aluminum ion (Al³⁺) and azide (N₃⁻) in aqueous acetonitrile solution via the fluorescence spectral changes. Stoichiometry and binding mechanisms for both sensors are well characterized and established by the respective spectroscopic techniques. L1 and L2 sensors are useful for the analysis of Al3+ and N3− in environmental samples and biological studies

### BACKGROUND OF THE INVENTION

### FIELD OF THE INVENTION

The present invention relates to rhodamine Schiff base compounds for the detection of micromolar levels of Al³⁺ ions and azide (N₃⁻).

### DESCRIPTION OF THE BACKGROUND

Several approaches discuss detection of aluminum but not in conjunction with azide. U.S. Pat. No. 9,891,237 relies on a Schiff base for metal cation detection, but its sensor relies on a form of benzazole. U.S. Pat. No. 7,615,377 also uses ligands for detection of metal ions, and it also is based on fluorescence, but it does not apply the specific formula towards the same specific ligands. U.S. Pat. No. 7,906,320 covers a fluorescence-based biosensor that can specifically detect metals and also discusses quenchers that emit at specific wavelength ranges. U.S. Pat. No. 7,018,840 refers to fluorescent metal sensors, with rhodamine complexed with metal ions through ligand binding but does not list aluminum as one of the exemplary metal ions. U.S. Pat. No. 5,567,619 detects for aluminum, among other elements/compounds and does mention some other similar attributes, such as chelation and certain color indications, but it is overall more primitive in nature.

### SUMMARY OF THE INVENTION

The present invention relates to sensor compounds (“sensors”) that are developed from rhodamine derivatives that may be used for detecting the presence of Al³⁺ and other metals.

Widespread use of aluminum in pharmaceuticals, cooking utensils, aluminum foil, vessels, and trays can result in the moderate increase in Al³⁺ concentration in food, and potentially damage the central nervous system in humans.

Novel and unobvious rhodamine Schiff base sensors L₁ and L₂ are described herein that are able to detect micromolar levels of Al³⁺ ions by the chelation-enhanced fluorescence (CHEF) process. Also of note, Al³⁺ complexes L₁-Al³⁺ and L₂-Al³⁺ behave as highly selective chemosensors for N₃⁻ ions by quenching of the fluorescence in acetonitrile/water (CH₃CN/H₂O) medium at 25° C.

The rhodamine derivative sensors L₁ and L₂ bearing 2-methoxy-1-naphthaldehyde and 5-bromo-3-methoxy salicylaldehyde units were designed and synthesized from the parent rhodamine B and aromatic aldehydes in a two-step Schiff base condensation, using microwave-assisted organic synthesis (MAOS) and utilized towards sequential fluorescence detection of aluminum ion (Al³⁺) and azide (N₃⁻) in aqueous acetonitrile solution. Aluminum ion (Al³⁺) triggers the formation of highly fluorescent ring-open spirolactam.

A mixture of ethanol with compound 2 and 2-methoxy-1-naphthaldehyde or with compound 2 and 5-bromo-3-methoxy salicylaldehyde was placed in a reaction vial and then stirred before being placed in a biotage microwave reactor. The closed reaction vessel in both cases was run under pressure and irradiated for 10 minutes. After cooling to room temperature, the resulting solid was filtered and washed three times with cold ethanol. After drying, the resulting sensor yield was measured—the L₁ sensor yielded 92%, while the L₂ sensor yielded 88%.

Absorption spectra studies showed that on incremental addition of Al³⁺ ions, the absorption intensity at 315 nm increased gradually and a new absorption peak at 565 nm with a shoulder at 525 nm was generated by ring opening with a visual color change from colorless to pink. The well-defined isosbestic points at 340 and 375 nm clearly indicates the formation of a new complex species between L₁ and Al³⁺. Absorption spectra of sensors recorded with the continuous addition of Al³⁺ showed a continuous increase in the absorption at 565 nm and that was employed to calculate binding constants for L₁ and L₂ with Al³⁺ using the Benesi-Hildebrand method.

The plot of absorbance of L₁ at 565 nm as a function of mole fraction of added Al³⁺ metal ion reveals that these probes bind to the metal ion in 1:1 stoichiometry. The fluorescence spectrum of sensors L₁ and L₂ showed a peak at 585 nm upon the addition of Al³⁺ corresponding to the delocalization in the xanthenes moiety of rhodamine.

The fluorescence and colorimetric response of the L₁-Al³⁺ and L₂-Al³⁺ complexes were quenched by the addition of N₃⁻, which extracted the Al³⁺ from the complexes and turned off the sensors, confirming that the recognition process is reversible. The recognition ability of the sensors was confirmed by fluorescence titration, Job's plot, 1H-NMR spectroscopy and density functional theory (DFT) calculations.

When L₁-Al³⁺ is used as the sensor for N₃⁻, high concentration of CN— interference must be eliminated by using mesoporous carbon based adsorbent. The addition of N₃⁻ to the L₁-Al³⁺ solution led to a change in color of the solutions from pink to colorless, which was observed with the naked eye. The addition of N₃⁻ to the solution containing L₁-Al³⁺ complex resulted in the reversal of the Al³⁺ induced changes in the emission band at 585 nm in the fluorescence emission spectra.

Gradual addition of N₃⁻ results in continuous decrease in the emission intensity at 585 nm. Based on fluorescence data, the detection limit of L₁-Al³⁺ or N₃⁻ was calculated as 12 μM. A similar finding was observed for complex L₂-Al³⁺ towards N₃⁻ ions. The L₂-Al³⁺ system revealed remarkably selective fluorescence “off” behavior exclusively with N₃⁻. The limit of detection value for N₃⁻ ions was found at 18 μM. These results show that L₁-Al³⁺ and L₂-Al³⁺ binds N₃⁻ ions with higher selectivity and the process is reversible.

Accordingly, there is provided according to an embodiment of the invention, a compound having the formula:

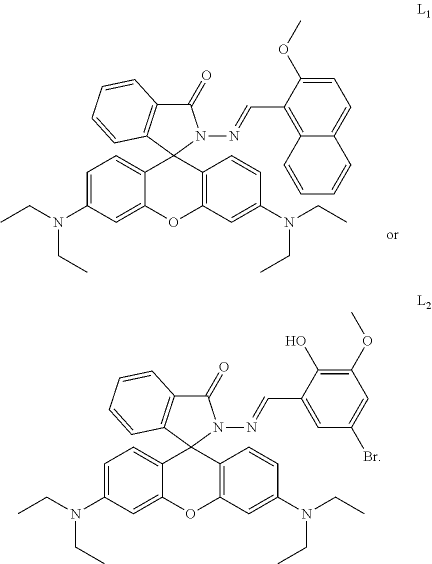 [SMILES: CCN(CC)c1ccc2c(c1)Oc1cc(N(CC)CC)ccc1C21c2ccccc2C(=O)N1/N=C/c1c(OC)ccc2ccccc12 ; CCN(CC)c1ccc2c(c1)Oc1cc(N(CC)CC)ccc1C21c2ccccc2C(=O)N1/N=C/c1cc(Br)cc(OC)c1O]

There is further provided according to the invention a compound having the formula:

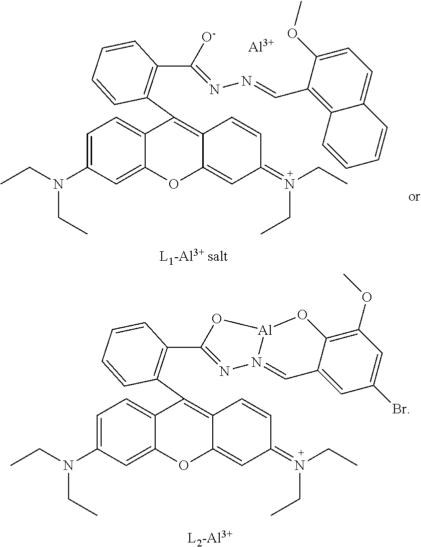

There is further provided according to the invention a method for synthesizing the compound L₁, comprising

mixing a compound having the formula

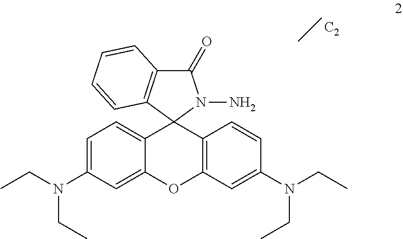

with 2-methoxy-1-naphthaldehyde and ethanol, stirring a resulting mixture until homogenous, and irradiating the resulting mixture in a microwave reactor.

There is further provided according to the invention a method for synthesizing the compound method for synthesizing the compound L2, comprising mixing a compound having the formula

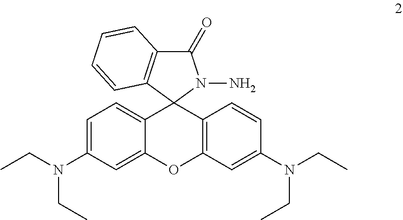 [SMILES: CCN(CC)c1ccc2c(c1)Oc1cc(N(CC)CC)ccc1C21c2ccccc2C(=O)N1N]

with 5-bromo-3methoxy salicylaldehyde and ethanol, stirring a resulting mixture until homogenous, and irradiating the resulting mixture in a microwave reactor.

There is further provided according to the invention a method for determining a presence of Al³⁺ in a sample, comprising: contacting the sample with a colorless solution comprising compound L₁ or L₂ and observing whether the colorless solution turns pink in color, where a change in color of the solution to pink indicates the presence of Al³⁺ in the sample. According to a further embodiment of the invention, the colorless solution shows no absorption above 450 nm in UV-vis absorption spectra, and an absorption peak above 525 nm indicates the presence of Al³⁺ in the sample.

There is further provided according to the invention a method for determining a presence of N₃⁻ in a sample, comprising: contacting the sample with a pink solution comprising a compound having the formula

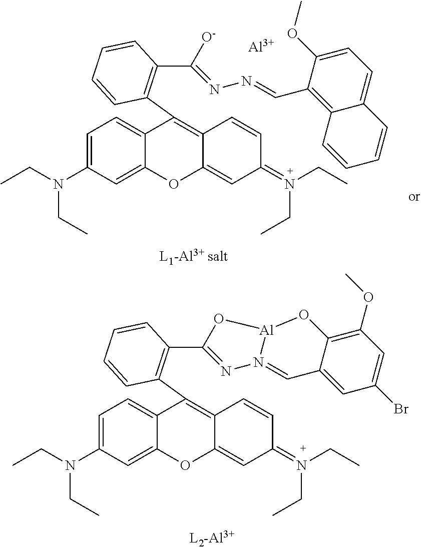

and observing whether the pink solution turns colorless, where a change in color of the solution from pink to colorless indicates the presence of N₃⁻ in the sample. According to a further embodiment of the invention, the pink solution shows an absorption peak above 525 nm in UV-vis absorption spectra, and no absorption above 450 nm indicates the presence of N₃⁻ in the sample.

### BRIEF DESCRIPTION OF THE DRAWINGS

FIG. 1 shows the chemical structures and synthetic routes of L₁ and L₂.

FIG. 2 shows UV-vis spectra of L₁ (10 μM) with Al³⁺ (0-23 μM) in CH₃CN/H₂O (7:3 v/v) solution.

FIG. 3 shows an Absorbance Job's plot for determination of L₁-Al³⁺ complex (10 μM) in CH₃CN/H₂O (7:3 v/v) solution.

FIG. 4a is a fluorescence spectra of L₁ (10 μM) in CH₃CN/H₂O (7:3 v/v) solution (λₑₓ=510 nm).

FIG. 4b is a fluorescence spectra of L₂ (10 μM) with metal ions (10 μM) in CH₃CN/H₂O (7:3 v/v) solution (λₑₓ=510 nm).

FIG. 5 is a fluorescence spectral titration of L₁ (10 μM) on the incremental addition of Al(NO₃)₃ (23 equivalents)(λₑₓ=510 nm).

FIG. 6 shows the effect of pH on fluorescence intensity of sensors L₁ and L₂ (10 μM).

FIG. 7a shows a proposed binding mechanism of sensor L₁ towards Al³⁺ in the presence and absence of azide (N₃⁻).

FIG. 7b shows a proposed binding mechanism of sensor L₂ towards Al³⁺ in the presence and absence of azide (N₃⁻).

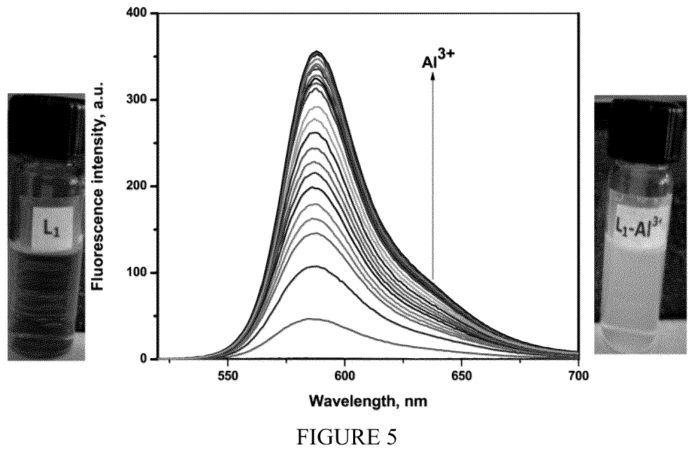

FIG. 5a shows the fluorescence spectra of L₁-Al³⁺ (1:1) with anions (10 μM) (λₑₓ=510 nm).

FIG. 8b shows a fluorescence spectral titration of L₁-Al³⁺ (23 equivalents of Al³⁺) on the incremental addition of N₃₋ (up to 35 equivalents) (λₑₓ=510 nm).

FIG. 9 shows ¹H-NMR spectral changes of L₂ (8 mM) in DMSO-d₆ and titrated with 0-1.0 equivalents of Al³⁺ in deuterated water.

FIG. 10a shows optimized structures and energy correlation of the HOMO-LUMO gap between L₁ and L₁-Al³⁺ salt.

FIG. 10b shows optimized structures and energy correlation of the HOMO-LUMO gap between L₂ and L₂-Al³⁺ complex.

### DETAILED DESCRIPTION

### Chemicals and Instruments

All reagents and solvents were purchased as analytical-grade and used without further purification unless otherwise stated. Stock solutions of metal ions were prepared from their nitrate and chloride salts and anion species from their tetrabutylammonium salts. Distilled deionized water was used throughout the experiments. ¹H-NMR and ¹³C-NMR spectra were recorded using an Avance 400 MHz spectrometer (BRUKER® Billerica, Karlsruhe, Germany) with tetramethylsilane (TMS) as internal standard and deuterated chloroform (CDCl₃) as solvent. NMR spectra were analyzed using MestReNova software (version 10, Mestrela Research, Feliciano Barrera-Bajo, Spain). The IR spectrum was obtained using FT-IR spectrometer (Shimadzu, IRAffinity-1S, Columbia, MD, USA). High resolution electrospray ionization mass spectrometry (ESI-MS) was acquired with a BRUKER® Apex-Qe instrument. All UV-vis spectroscopy experiments were recorded using a Cary UV/vis spectrophotometer 5000 (VARIAN®, Walnut Creek, CA, USA). Fluorescence emission spectra experiments were measured using a Cary 60 series spectrometer (AGILENT®, Walnut Creek, CA, USA), with excitation and emission slit widths of 5 nm and excitation wavelength at 510 nm. MAOS reactions were carried out in a single mode BIOTAGE® Initiator 2.0 (BIOTAGE®, Uppsala, Sweden).

### Microwave-Assisted Synthesis and Characterization of L₁ and L₂

Sensors L₁ and L₂ were synthesized from the parent rhodamine B and aromatic aldehydes (2-methoxy-1-naphthaldehyde and 5-bromo-3-methoxy salicylaldehyde) in a two-step Schiff base condensation using MAOS heating protocols, as shown in FIG. 1. Compound 2 was synthesized according to procedure reported in Xiang Y, Tong A, Jin P, Ju Y, Org. Lett 2006, 8, 2863.

### Synthesis of Sensor L₁

Using microwave heating protocol: A mixture of compound 2 (105 mg, 0.230 mmol), 2-methoxy-1-naphthaldehyde (41 mg, 0.220 mmol) and ethanol (2 ml) was placed in a 10 ml reaction vial. The resulting mixture was stirred to make it homogeneous and it was placed in the cavity of a biotage microwave reactor. The closed reaction vessel was run under pressure and irradiated for 10 min at 100° C. After cooling to room temperature, the resulting solid was filtered and washed three times with cold ethanol. After drying, the sensor L₁ was isolated to give in 92% yield. Melting point: 244-246° C.; ¹H-NMR (CDCl₃), δ (ppm): 9.63 (1H, s, N=C—H); 8.77 (1H, t, J=7.4 Hz, H—Ar), 7.74 (1H, d, J=8.4 Hz, H—Ar), 7.71 (1H, d, J=8.0 Hz, H—Ar), 7.63 (1H, d, J=7.7 Hz, H—Ar), 7.48-7.51 (2H, m, H—Ar), 7.15-7.27 (2H, m, H—Ar), 7.12 (1H, d, J=8.4 Hz), 7.09 (1H, d, J=4.9 Hz), 6.63 (2H, d, J=8.8 Hz), 6.44 (2H, d, J=2.2 Hz), 6.28 (2H, dd, J=8.8 Hz, 2.6 Hz), 3.82 (3H, s, OCH₃), 3.31 (8H, q, J=6.9 Hz, NCH₂CH₃), 1.14 (12H, t, J=6.9 Hz, NCH₂CH₃). ¹³C-NMR (CDCl₃), δ(ppm): 164.6, 157.8, 153.4, 151.7, 148.8, 147.6 (N═C—H), 137.6, 133.1, 131.9, 130.3, 129.2, 128.1, 127.0, 126.7, 124.0, 123.2, 116.8, 112.9, 108.1, 107.9, 106.5, 104.6, 79.9, 66.3 (spiro carbon), 56.7, 44.3 (NCH₂CH₃), 12.7 (NCH₂CH₃); HRMS (ESI): m/z calcd for C₄₀H₄₀N₄O₃: 625.3173; Found: 625.3176 [M+H]+.

### Synthesis of Sensor L₂

Using microwave heating protocol: A mixture of compound 2 (100 mg, 0.220 mmol), 5-bromo-3-methoxy salicylaldehyde (51 mg, 0.221 mmol) and ethanol (2 ml) was placed in a 10 ml reaction vial. The resulting mixture was stirred to make it homogeneous and it was placed in the cavity of a biotage microwave reactor. The closed reaction vessel was run under pressure and irradiated for 10 min at 100° C. After cooling to room temperature, the resulting solid was filtered and washed three times with cold ethanol. After drying, the sensor L₂ was isolated to give in 88% yield. ¹H-NMR (CDCl₃), δ (ppm): 11.11 (1H, s, —OH), 8.94 (1H, s, —CH═N), 7.96 (¹H, t, J=6.6 Hz, —Ar), 7.49 (2H, m, —Ar), 6.86 (1H, d, J=6.6 Hz, —Ar), 7.50 (2H, s, —Ar), 6.51-6.43 (4H, m, —Ar), 6.25 (2H, d, J=7.5 Hz, —Ar), 3.82 (3H, s, —OCH₃), 3.31 (8H, q, NCH₂CH₃), 1.16 (12H, t, J=6.6 Hz, NCH₂CH₃) 13C-NMR (CDCl₃), δ (ppm): 163.6, 152.7, 148.5, 146.6 (—CH═N), 138.5, 138.1, 137.7, 134.0, 128.9, 128.5, 127.5, 123.1, 121.8, 121.3, 108.1, 108.0, 106.5, 104.8, 97.3, 80.9, 65.5 (spiro carbon), 56.1, 43.6 (NCH₂CH₃), 12.4 (NCH₂CH₃). HRMS (ESI): m/z calcd for C₃₆H₃₇BrN₄O₄: 669.2071; Found: 669.2076 [M+H]+.

### General Procedure for the Spectroscopic Studies

All spectroscopic measurements were carried out in aqueous CH₃CN medium at room temperature. Stock solutions of sensors L₁ and L₂ (1×10⁻³ M), selected salts of cations (1×10⁻³ M) and anions (1×10⁻⁴ M) were prepared in CH₃CN/H₂O. Thus, L₁-Al³⁺ and L₂-Al³⁺ solutions for N₃⁻ detection were prepared by addition of 1.0 equivalent of Al³⁺ to the solution of both L₁ and L₂ (20 μM) in Tris-HCl (10 mM, pH=7.2) buffer containing CH₃CN/H₂O (7:3, v/v) solution. The resulting solution was shaken well before recording the spectra. Each fluorescence titration was repeated at least thrice until consistent values were obtained. Jobs continuous variation method was used for determining the binding stoichiometry of the complexation reaction. The association constant (K) was calculated from absorbance studies by the linear Benesi-Hildebrand equation. Color changes in solution phase were observed visually under normal light and under a hand-held UV lamp upon addition of various metal ions at room temperature.

### Synthesis of Sensors L1 and L2

The synthesis of L₁ and L₂ were prepared in two steps with 92% and 88% overall yields respectively (FIG. 1). The results obtained indicate that, unlike classical heating, MAOS results in higher yields, shorter reaction time, mild reaction condition, simple work-up procedure and better purity offer privilege over other methods where complex chromatographic techniques are required for purification of the target compounds. The structure of sensors was fully characterized by ¹H-NMR, ¹³C-NMR, FT-IR and HRMS spectroscopy and all data are in accordance with the proposed structure.

### Absorption Spectra Studies

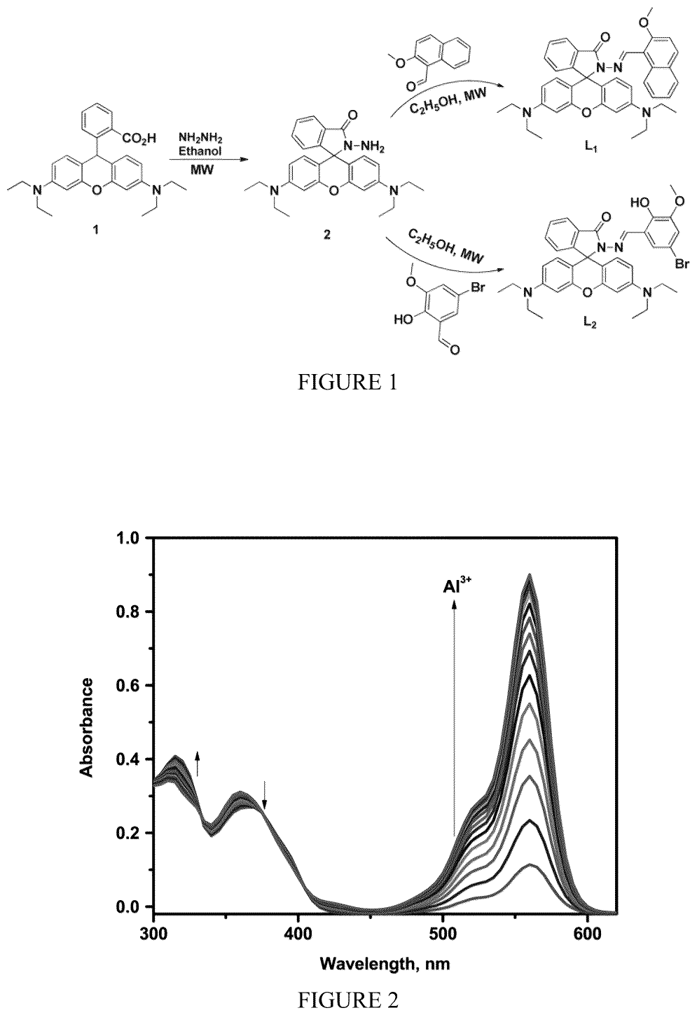

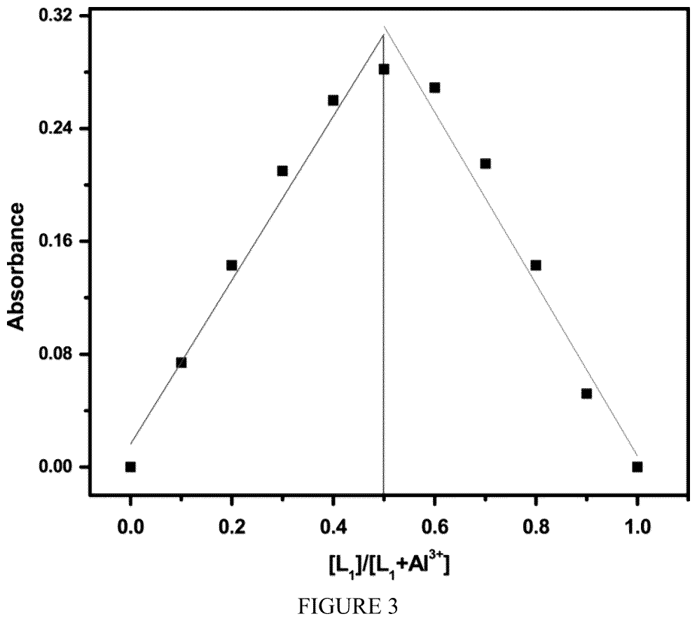

The metal ion sensing of L₁ and L₂ were first investigated by UV-vis absorption spectra. The colorless solutions were very weakly fluorescent and showed no absorption above 450 nm, properties which are characteristic of the predominant ring-closed spirolactam. The predominant spirolactam form was further confirmed by observation of the characteristic carbon resonance near 66 ppm for each of the sensors. The UV-vis spectra of sensors were recorded in buffer at 25° C. and showed an absorption maximum at λ=315 nm, which may be attributed to the intramolecular π-π* charge transfer transition. On incremental addition of Al³⁺ ions, the absorption intensity at 315 nm increased gradually and a new absorption peak at 565 nm with a shoulder at 525 nm was generated by ring opening with a visual color change from colorless to pink. The well-defined isosbestic points at 340 and 375 nm clearly indicates the formation of a new complex species between L₁ and Al³⁺ ion (FIG. 2). The absorption enhancement is high compared to other metal ions. Selectivity of Li was checked in the presence of other metal ions. No significant change in the UV-vis spectrum was observed upon the addition of a 10 equivalent excess of other metal ions of interest: Na⁺, K⁺, Mg²⁺, Ca²⁺, Ni²⁺, Zn²⁺, Co²⁺, Hg²⁺, Pb²⁺, Fe2+, Fe³⁺, Cr²⁺ and Cu²⁺. Absorption spectra of sensors recorded with the continuous addition of Al³⁺ showed a continuous increase in the absorption at 565 nm and that was employed to calculate binding constants for L₁ and L₂ with Al³⁺ using the Benesi-Hildebrand method. The plot of absorbance of L₁ at 565 nm as a function of mole fraction of added Al³⁺ metal ion reveals that these probes bind to the metal ion in 1:1 stoichiometry (FIG. 3). The complex association constant (K) calculated through the Benesi-Hildebrand equation for Al³⁺ with L₁ and L₂ were found to be 3.82×104 M⁻¹ and 2.41×104 M⁻¹, respectively.

### Fluorescence Spectral Response of Sensors

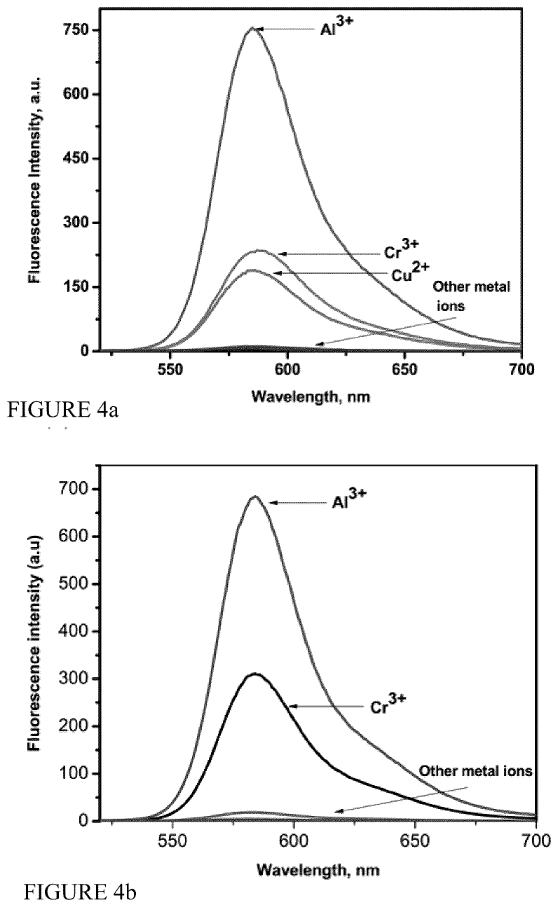

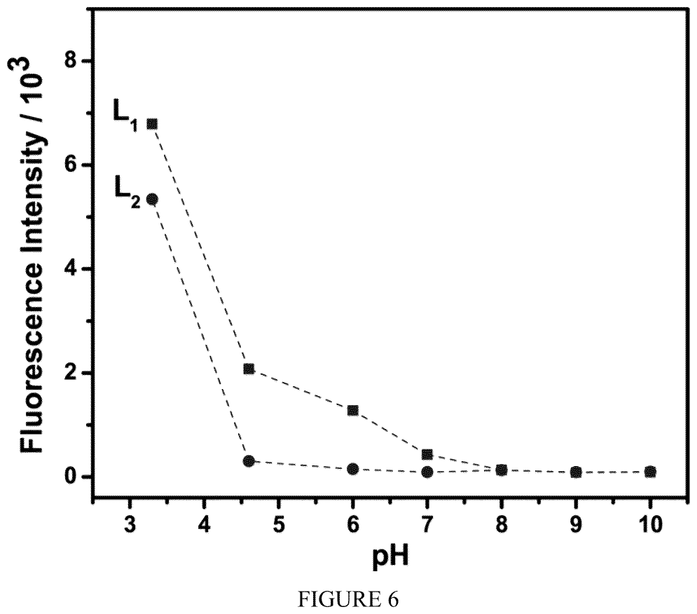

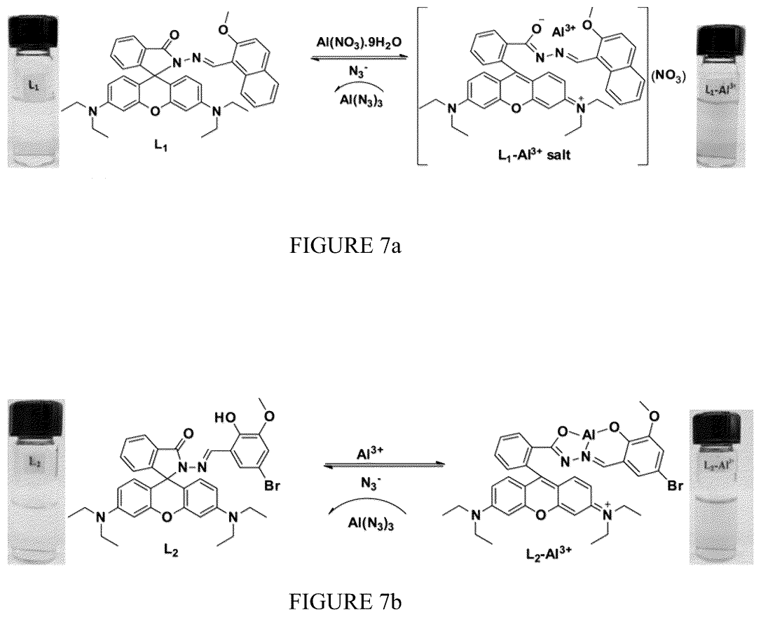

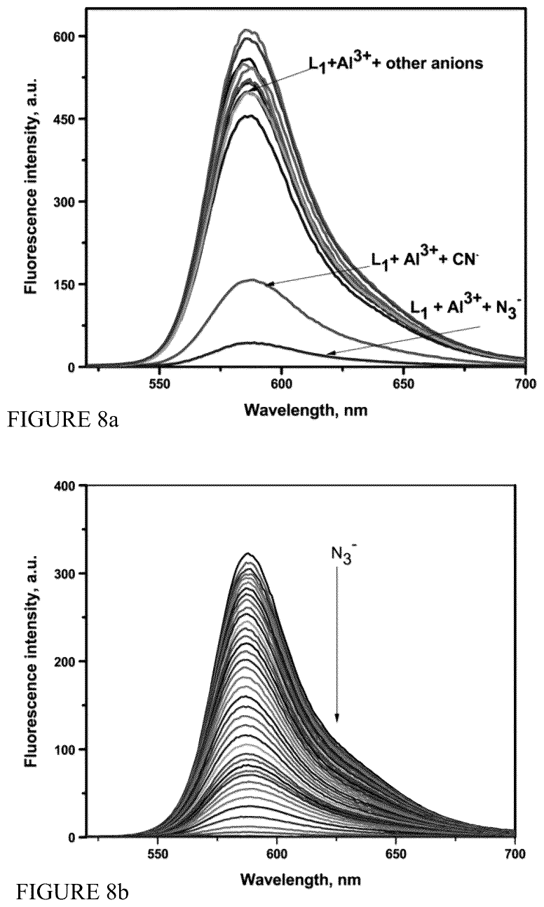

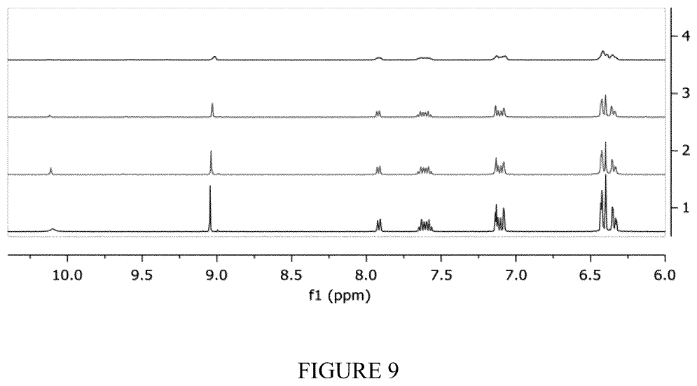

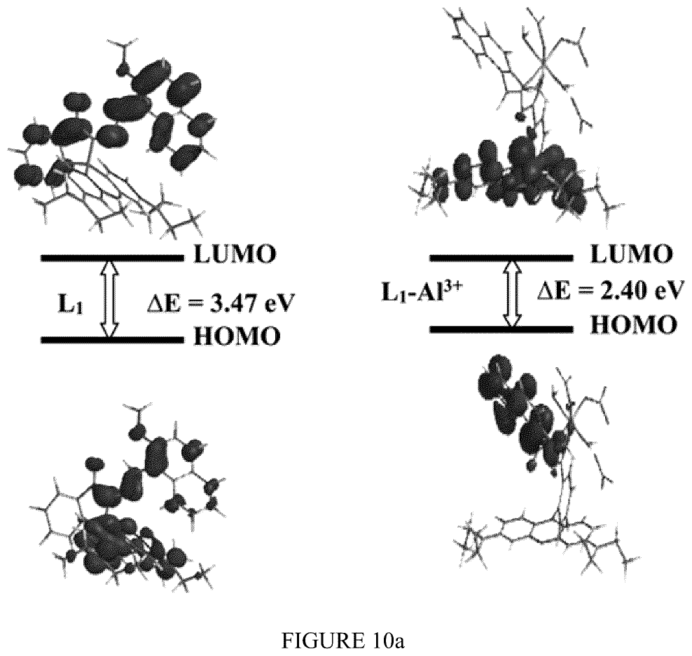

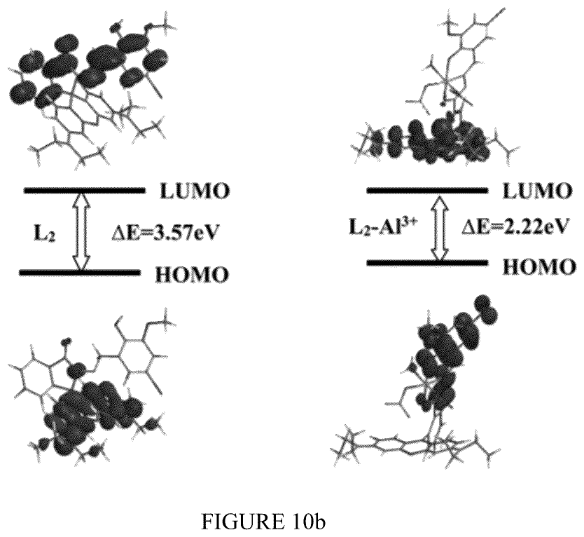

To further explore the sensing behavior of L₁ for Al³⁺ ion, the fluorescence spectra of L₁ in CH₃CN with various metal ions were examined. The fluorescence spectra were obtained by excitation at 510 nm, and both the excitation and emission slit were 5 nm. The fluorescence intensity of L₁ upon the additions of metal ions in CH₃CN showed a remarkable sensitivity and selectivity towards Al³⁺, even though there were relatively small effects with Cu²⁺ and Cr³⁺ (FIG. 4a). There was a significant emission intensity enhancement with 1.0 equivalent of Al³⁺ which indicates sensor L₁ is an excellent turn-on sensor for Al³⁺. This very high fluorescence enhancement is attributed to the formation of ring-open spirolactum in the presence of Al³⁺. This selectivity for Al³⁺ ions over all other ions is due to selective chelate formation with L₁ to afford an L₁-Al³⁺ complex (See FIG. 7a). When illuminated with a hand-held UV lamp, the addition of Al³⁺ ions to sensor solution resulted in orange fluorescence emission from L₁ solution (FIG. 5). The fluorescence profile of L₂ were very similar to those for sensor L₁: again Al³⁺ registered the highest fluorescence enhancement while other metal ions showed no significant enhancement (FIG. 4b). The fluorescence spectrum of sensors L₁ and L₂ showed a peak at 585 nm upon the addition of Al³⁺ corresponding to the delocalization in the xanthenes moiety of rhodamine. It is assumed that the spirolactam form was opened upon the addition of Al³⁺ to sensors and makes a highly delocalized x-conjugated stable complexes with Al³⁺ through their active donor sites (e.g., N and O atoms) of receptor part, though other ions failed which basically indicates that the coordinate moiety of L₁ and L₂ matches perfectly with Al³⁺ ions instead of the other ions. The detection limits of L₁ and L₂ for Al³⁺ ions were estimated based on the fluorescence titration experiment as 32 μM and 47 μM respectively. Furthermore, the effect of pH values on the fluorescence of L₁ and L₂ were also investigated in a pH range from 3 to 10. FIG. 6 shows that for free L₁ and L₂ at pH<5, due to protonation of the open-ring of spirolactam, an obvious color change and fluorescence turn-on appeared. Thus, all the optical measurements were performed in buffer solution with a pH of 7 to keep the sensors in their ring closed form.

### Detection of Azide (N₃⁻)

Investigation of the reversible binding nature of the sensors is shown in FIG. 8 and FIGS. 7a and 7b. Due to the high stability of AlN₃, the L₁-Al³⁺ and L₂-Al³⁺ complexes serve as a means to detect N₃⁻. FIG. 8a shows the addition of 20 μM of anions N₃⁻, CN⁻, ClO₄⁻, CH₃COO⁻, HSO₄⁻, H₂SO₄²⁻, SCN⁻, Cl⁻, I⁻, F⁻, and OH⁻ to L₁-Al³⁺ (1:1) of which N₃⁻ alone quenches the fluorescence, with a slight effect for CN, indicating high selectivity for N₃⁻. High concentration of CN⁻ contamination is likely to mislead the fluorescent selectivity of N₃⁻. So, when L₁-Al³⁺ is used as the sensor for N₃⁻, high concentration of CN⁻ interference must be eliminated by using mesoporous carbon based adsorbent. The addition of N₃⁻ to the L₁-Al³⁺ solution led to a change in color of the solutions from pink to colorless, which was observed with the naked eye. The addition of N₃⁻ to the solution containing L₁-Al³⁺ complex resulted in the reversal of the Al³⁺ induced changes in the emission band at 585 nm in the fluorescence emission spectra. Gradual addition of N₃⁻ results in continuous decrease in the emission intensity at 585 nm (FIG. 8b). Based on fluorescence data, the detection limit of L₁-Al³⁺ for N₃⁻ was calculated as 12 μM. A similar finding was observed for complex L₂-Al³⁺ towards N₃⁻ ions. The L₂-Al³⁺ system revealed remarkably selective fluorescence “off” behavior exclusively with N₃⁻. The limit of detection value for N₃⁻ ions was found at 18 μM. These results show that L₁-Al³⁺ and L₂-Al³⁺ bind N₃⁻ ions with higher selectivity and that the process is reversible. The expected binding mechanism of sensors with Al³⁺ in the presence and absence of azide (N₃⁻) is shown in FIGS. 7a and 7b.

### FT-IR and ¹H-NMR Study for Elucidation of Coordination Mechanism Between Sensors and Al³⁺

To elucidate the coordination mechanism of L₁-Al³⁺ and L₂-Al³⁺ complexes, the FT-IR spectrum of L₁ and L₂ were conducted in the absence and presence of Al³⁺ ion. The characteristic peak of the amide carbonyl γ(c=o) shifted from 1680 cm⁻¹ to 1614 cm⁻¹ in the presence of Al³⁺, indicating that carbonyl O atoms of the L₁ and L₂ are involved in the coordination of Al³⁺. ¹H-NMR was also performed by adding Al³⁺ to deuterated dimethyl sulfoxide (DMSO-d₆) solution of L₂ as shown in FIG. 9. The L₂-Al³⁺ complexes were prepared by the additions of 0.25, 0.5 and 1.0 equivalent AlCl₃·6H₂O to the DMSO solution of L₂. The peaks observed at δ 10.10 and δ 9.07 are attributable to the phenolic OH and the imine proton (—CH═N—) in L₂. Addition of 1 equivalent of Al³⁺ resulted in the disappearance of the hydroxyl proton indicating the binding of Al³⁺ ion through the phenoxide interaction. Further, small unfilled-shifts from 9.07 to 9.00 ppm and shortening of imine protons were observed because of the complex formation between nitrogen atoms and Al³⁺. The formation of the L₂-Al³⁺ complex through normal ring opening was confirmed by performing the ¹³C-NMR experiment with L₂ in the absence and presence of Al³⁺ ions, from which it was observed that the signal at δ=66 ppm attributable to the tertiary carbon of the spirolactam ring in L₂ was absent from the spectrum of L₂-Al³⁺ complex. Therefore, it is understood that the O atom of phenolic OH, N atom of imine and O atom of spiro ring coordinate to Al³⁺ as shown in FIGS. 7a and 7b.

### Geometry Optimization

To better understand the nature of the coordination of Al³⁺ with sensors, theoretical calculations on structures L₁, L₂, L₁-Al³⁺ and L₂-Al³⁺ were carried out using Spartan' 16 software. Density functional theory (DFT), employing the B3LYP functional and the 6-31G* basis set was used to obtain gas phase, optimized geometries of these structures. The optimized structures of L₁, L₂ and their respective Al-complexes are depicted in FIGS. 10a and 10b. L₁ and L₂ can undergo rotation of approximately 180° about the N—N bond, producing two prominent cis and trans conformations. For both L₁ and L₂, the trans conformation is more energetically stable than the respective cis one by approximately 11.3 kJ mol⁻¹, owing to anti-arrangement of the methoxy (—OMe) group and the xanthene moiety in trans L₁ and to the anti-arrangement of the hydroxyl (—OH) group and the xanthene moiety in trans L₂. Additionally, in trans L₁ the energy gap between the highest occupied molecular orbital (HOMO) (−4.81 eV) and the lowest unoccupied molecular orbital (LUMO) (−1.34 eV) is 3.47 eV, and in cis L₁ the gap, HOMO (−5.03 eV) and LUMO (−1.35 eV), is 3.68 eV. In trans L₂, the energy gap, HOMO (−4.86 eV) and LUMO (−1.29 eV) is 3.57 eV, and in cis L₂ the energy gap, HOMO (−5.05 eV) and LUMO (−1.22 eV) is 3.83 eV, suggesting that trans L₁ and trans L₂ are the major equilibrium conformations available stereochemically for direct Al³⁺ coordination. Also, in trans L₁, the electron density is delocalized over the entire xanthene moiety with some found on the spirolactam ring as well as on the imine and the ortho-methoxy naphthalene moieties (FIG. 10a). In cis L₁, the electron density is mainly localized on half of the xanthene moiety. In both trans L₂ and cis L₂, the electron density is mainly located over the entire xanthene moiety with some found on the lactam ring nitrogen of both. Moreover, some electron density is also found on the carbonyl oxygen in trans L₂ but not on the carbonyl oxygen in cis L₂ (FIG. 10b).

Density functional calculations of molecular interactions of trans-L₁ and trans-L₂ with aqueous aluminum (Al³⁺) nitrate solution revealed that both sensors are energetically stabilized on binding with Al³⁺ ions. For instance, upon formation of L₁-Al³⁺ salt complex, the HOMO-LUMO energy gap in trans-L₁(ΔE=3.47 eV) decreased to ΔE=2.40 eV, and upon formation of L₂-Al³⁺ complex, the HOMO-LUMO energy gap in trans-L₂(ΔE=3.57 eV) decreased to 2.22 eV. In L₁-Al³⁺ salt complex, formulated as [Al(L₁)NO₃)₂(H₂O)₂][NO₃], HOMO is primarily delocalized over the methoxy naphthalene moiety, while LUMO is primarily delocalized over the xanthene moiety. In L₂-Al³⁺ complex, formulated as Al(L₂)(NO₃)₂(H₂O), HOMO is found over the tricyclic structure about Al³⁺ while LUMO is delocalized over the xanthene moiety (FIGS. 10a and 10b).

Vertical electronic excitations of optimized B3LYP/6-31G* trans-L₁, trans-L₂ and their respective complexes were computed using time-dependent-density functional theory (TD-DFT) Spartan'16 software calculations, formalized in water and using a conductor-like polarizable continuum model (CPCM). In the TD-DFT UV-vis spectrum of trans-L₁, an absorption band at λ=379.24 nm with a vertical excitation energy of 3.2693 eV and corresponding to HOMO-2→LUMO excitation (oscillator strength=0.4632) dominates. While in the TD-DFT UV-vis spectrum of trans-L₁-Al³⁺ salt complex, an absorption band at λ=422.57 nm dominates, corresponding to HOMO→LUMO excitation (vertical excitation energy=2.9341 eV and oscillator strength=1.0951). In the case of trans-L₂, an absorption band at λ=344.32 nm dominates, corresponding to HOMO-2→LUMO excitation with a vertical excitation energy of 3.6008 eV and an oscillator strength=0.3152. For trans-L₂-Al³⁺ complex, an absorption band at λ=456.19 nm dominates, corresponding to HOMO-1→LUMO and HOMO→LUMO excitations with a vertical excitation energy of 2.7178 eV and an oscillator strength=0.7824.

### Conclusion

We have developed reversible fluorescent sensors L₁ and L₂ for the selective and sensitive sequential detections of Al³⁺ and N₃⁻ via the fluorescence spectral changes. Upon binding to Al³⁺, obvious detectable change in fluorescence was observed due to the CHEF effect. The in situ prepared L₁-Al³⁺ and L₂-Al³⁺ complexes were used to detect N₃⁻ via the metal-displacement approach which displayed an excellent selectivity and sensitivity towards N₃⁻. Thus, upon the addition of N₃⁻ to complexes, the intensity of the 585 nm band decreases, indicating release of L₁ and L₂ from the aluminum complexes. Stoichiometry and binding mechanisms for both sensors are well characterized and established by the respective spectroscopic techniques. These results clearly demonstrate that L₁ and L₂ sensors described herein will be useful for the analysis of Al³⁺ and N₃⁻in environmental samples and biological studies.

1. A compound having the formula:

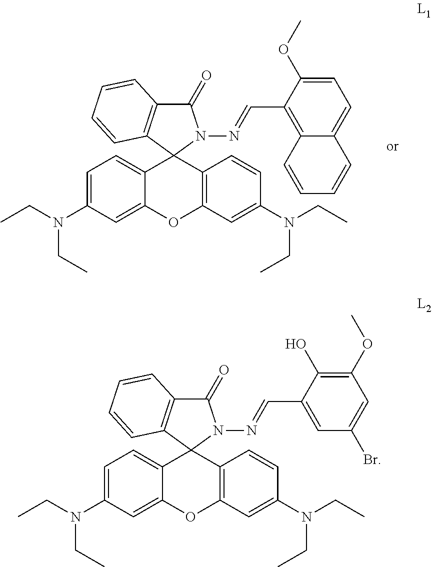 [SMILES: CCN(CC)c1ccc2c(c1)Oc1cc(N(CC)CC)ccc1C21c2ccccc2C(=O)N1/N=C/c1c(OC)ccc2ccccc12 ; CCN(CC)c1ccc2c(c1)Oc1cc(N(CC)CC)ccc1C21c2ccccc2C(=O)N1/N=C/c1cc(Br)cc(OC)c1O]

2. A compound having the formula:

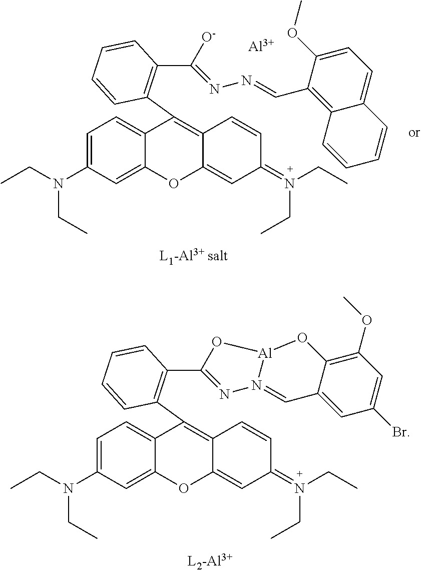

3. A method for synthesizing the compound L₁ of claim 1, comprising mixing a compound having the formula 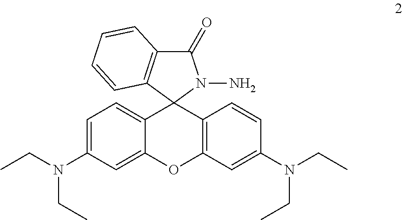 [SMILES: CCN(CC)c1ccc2c(c1)Oc1cc(N(CC)CC)ccc1C21c2ccccc2C(=O)N1N]
with 2-methoxy-1-naphthaldehyde and ethanol, stirring a resulting mixture until homogenous, and irradiating the resulting mixture in a microwave reactor.

4. A method for synthesizing the compound L₂ of claim 1, comprising mixing a compound having the formula 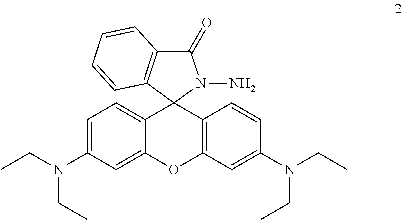 [SMILES: CCN(CC)c1ccc2c(c1)Oc1cc(N(CC)CC)ccc1C21c2ccccc2C(=O)N1N]
with 5-bromo-3methoxy salicylaldehyde and ethanol, stirring a resulting mixture until homogenous, and irradiating the resulting mixture in a microwave reactor.

5. A method for determining a presence of Al³⁺ in a sample, comprising:
contacting the sample with a colorless solution comprising a compound according to claim 1 and observing whether the colorless solution turns pink in color, where a change in color of the solution to pink indicates the presence of Al³⁺ in the sample.

6. A method for determining a presence of N₃⁻ in a sample, comprising:
contacting the sample with a pink solution comprising a compound according to claim 2 and observing whether the pink solution turns colorless, where a change in color of the solution from pink to colorless indicates the presence of N₃⁻ in the sample.

7. A method according to claim 5 wherein the colorless solution shows no absorption above 450 nm in UV-vis absorption spectra, and wherein an absorption peak above 525 nm indicates the presence of Al³⁺ in the sample.

8. A method according to claim 6 wherein the pink solution shows an absorption peak above 525 nm in UV-vis absorption spectra, and wherein no absorption above 450 nm indicates the presence of N₃⁻ in the sample.
